(19) United States
(12) Patent Application Publication
(10) Pub. No.: US 2007/0269490 A1
(43) Pub. Date: Nov. 22, 2007

(54) Material for Processed Food for Weight Reduction Diets and Weight Reduction Dietary Processed Food Using Thereof

(76) Inventors: Shigeru Nakajima, Kanagawa (JP); Masayuki Hashidume, Shizuoka (JP); Yoshinori Horaki, Shizuoka (JP); Takehisa Kurebayashi, Shizuoka (JP)

Correspondence Address: PEPPER HAMILTON LLP, ONE MELLON CENTER, 50TH FLOOR, 500 GRANT STREET, PITTSBURGH, PA, 15219, US

(21) Appl. No.: 10/597,920

(22) PCT Filed: Sep. 3, 2004
(86) PCT No.: PCT/JP04/12834

§ 371 Date: May 11, 2007

(30) Foreign Application Priority Data
Feb. 12, 2004 (JP) 2004-035767

(51) Int. Cl. A61K 47/00; A23J 3/04; A61K 35/60

(52) U.S. Cl. 424/439; 424/523

(57) ABSTRACT

The purpose of the invention is to provide a material for weight reduction diet food and the processed food using the same, that is a natural food product obtained from the materials eaten in everyday life, and very safe to the human body. There is provided a material for processed food for weight reduction diets, wherein active ingredients including histidine were extracted from Bonito essence. In addition, the present invention provides the material for processed food for weight reduction diets, wherein 12,000 mg-20,000 mg weight % of histidine is included. Furthermore, the processed food for weight reduction diets using the above-mentioned material for processed food for weight reduction diets is offered. As examples of such processed food, besides supplements in tablets and capsules soup stock powder, Furikake, and feeds for animals. etc, can be considered,

### CROSS-REFERENCE TO RELATED APPLICATIONS

This application claims priority under 35 U.S.C. 119 based upon Japanese Patent Application Serial No. 2004-035767, filed on Feb. 12, 2004. The entire disclosure of the aforesaid application is incorporated herein by reference.

### FIELD OF THE INVENTION

This invention is related to dietary processed foods and its raw materials, and more specifically to processed food for weight reduction diets and its raw materials, comprising extracted ingredients from fish, that are gentle and safe to the human body.

### BACKGROUND OF THE INVENTION

In recent years, obesity has become a serious problem in our country as our food culture becomes Westernized. Obesity is highly linked to life-style diseases (adult diseases), and the necessity of its prevention and relief has been strongly pointed out.

However, from the viewpoint of today's life-style, once a person becomes obese, it is not easy to lose weight. That is, in general, in order to increase the amount of energy consumption, direct physical exercise is effective. However, if the main purpose of exercise is weight loss, an intense workout is required. Thus it is not easy for busy-working men and women. Accordingly, there is an option of drug therapy as a way to prevent and relieve obesity without doing exercise, by just ingesting a drug. However, it is a method taken for severe obesity cases and not intended for general use, nor it is safe. Therefore, the demand for health food/supplements with a high weight-loss effect and little health hazard, which can be ingested in the course of normal daily life, is increasing.

Japanese Patent Application, Publication No. 2002-142723 can be referred to for such health food/supplements.

In the light of the above-mentioned problems, the present invention aims to provide a material for weight reduction diet food and the processed food using the same, which is a natural food product and obtained from materials that we eat normally and is safe to the human body.

### SUMMARY OF THE INVENTION

The present invention aims to solve the above-mentioned problems, and according to its principal aspect, an object of the invention is to provide a raw material for processed food for weight reduction diets, extracted from fish, and containing histidine, as an active ingredient.

In recent years, the understanding of the antiobesity effect of histidine (a food ingredient and an amino acid type) has advanced, unraveling its appetite-suppressing and fat-burning effects. (Histidine induces lipolysis through sympathetic nerve in white adipose tissue: European journal of Clinical Investigation Vol. 32, April 2002:236-241; Histidine suppresses food intake through its conversion into neuronal histamine: Experimental Biology and Medicine Vol. 227, January 2002:63-68; Intake Suppression Effect on Rats with freely-available Histidine added feed: Japan Society of the Study of Obesity, Vol. 8, No. 1, April 2002:55-60) also, epidemiologic research results that support these effects have also been reported (Suppression of Food Intake by Histidine-rich protein under Low Energy Intake; Japanese Society of Nutrition and Food Science Journal. Vol. 53,:2007-214 (2002); Co-relation between the energy intake and histidine intake among the women in Setonaikai area; Japan Society of the Study of Obesity, Vol. 7, No. 3: 72-78, December 2001).

Therefore, it is conceivable to employ histidine as a dieting supplement. However, while histidine itself is an ingredient of food products, thus being safe, general consumers are unfamiliar with it. Moreover, since it is a food additive, the possibility of its being accepted by the consumer is likely to be low.

After contemplating the above issues, the inventors of the present invention sought a generally well-known food that contains a high concentration of histidine, and came to a conclusion that a dietary supplement using such food will be accepted by consumers. Subsequently, the inventors fortunately discovered that fish, especially bonitos contains a high concentration of histidine, and moreover, Bonito essence that has been widely used as a natural flavor enhancer is also rich in histidine. In case of a weight reduction diet supplemental food using bonito, it gives a better impression to consumers than one using histidine itself which is a food additive. Moreover, since the histidine contained is 100% natural histidine from Bonito, it is safe for consumers.

However, with the modern dietary customs, it is difficult to consume bonito or bonito processed foods daily, such as Sashimi, dried bonito, Namari bushi (smoked bonito), and canned bonito. Therefore, the inventors came to the conclusion that it is appropriate to powderize a Bonito extract to create weight reduction diet processed food materials that can be added to other food, and, through analysis and experiments, they arrived at the confirmation of their weight loss effects, thus completing this invention.

According to an embodiment of the present invention, there is provided a material for processed food for weight reduction diets, wherein active ingredients including histidine are extracted from Bonito essence by membrane concentration.

Also, according to an embodiment of the present invention, there is provided a material for processed food for weight reduction diets, wherein 12,000 mg-20,000 mg weight percent of the histidine is contained.

Moreover, according to an embodiment of the present invention, there is provided a dietary processed food using the material for processed food for weight reduction diets. As examples of such processed foods, besides supplements in tablets and capsules, soup stock powder, Furikake, and feeds for animals, etc. can be considered.

### BRIEF DESCRIPTION OF THE DRAWINGS

FIG. 1 is a graph showing the amino acid composition of the material for processed food for weight reduction diets according to an example of the present invention.

FIG. 2 is a graph showing free amino acid composition of the material for processed food for weight reduction diets according to the example of the present invention.

FIG. 3 is a table showing the ingredients of the processed food for weight reduction diets (Bonimax food) related to the present invention; the control; and the high protein feed, that were used in this example.

FIG. 4 is a graph showing the relationship between the feed intake amounts and feeding periods in this example.

FIG. 5 is a graph showing comparison of all feed intakes.

FIG. 6 is a graph showing comparison of liver weights after the feeding period.

FIG. 7 is a graph showing comparison of endoabdominal fat after the feeding period.

FIG. 8 is a graph showing comparison of fats around testicles after the feeding period.

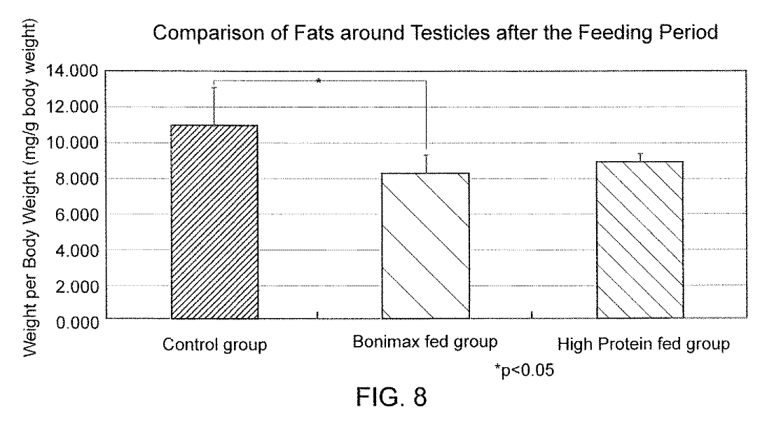

FIG. 9 is a graph showing a relationship between the number of feeding days and body weight change.

FIG. 10 is a graph showing a relationship of body weight increase.

### DETAILED DESCRIPTION OF ILLUSTRATIVE EMBODIMENTS

Hereafter, preferred embodiments and examples of the present invention are described in detail.

The natural flavoring materials extracted from fish, especially Bonito extract, are widely known as natural flavor enhancers containing a lot of amino acids, peptide, nucleic acids, and others that enhance flavors.

This bonito extract can be powderized to make tablets and capsules as is. However, large-size tablets tend to be avoided by consumers. Even with small tablets and capsules, intakes with a higher frequency tend to be avoided by them as well, thus not achieving the consumption with a required amount of histidine. In light of the above condition, the inventors decided to provide the present invention as a material for processed food for weight reduction diets that can be added to various food products, in addition to the above-described dietary supplements in medicinal forms.

On the other hand, the above-mentioned material has high salt content from bonito, since it is a highly condensed material of bonito extract. As a diet supplement that can be added to various foods, it is desirable to have as little salt content as possible. In addition, because Bonito essence is an extract of bonito, it has a characteristic fishy smell. For a dietary supplement that can be added to various foods, this smell has to be removed.

First activated charcoal was added to Bonito Essence A-45, made by Kabushikikaisha Maruhachi Muramatsu and stirred for deodorization. After the deodorization, the activated charcoal was removed, and histidine was concentrated using reverse osmosis membrane (for concentration), followed by a desalination process using electrodialysis membrane. The resultant product was powderized to create a material for processed food for weight reduction diets.

However, when Bonito essence is concentrated using the membrane, in general, the amino acids and nucleic acids, etc. that give the flavor of characteristic Bonito are also condensed. Thus, it is generally thought that it promotes appetite rather than suppressing appetite when this is added to food. Therefore, using the food product obtained as explained above for the raw material for processed food for weight reduction diets is not considered in general by those skilled in the art.

Therefore, the inventors fed rats for a period of one week with a rat feed mixed with the material for processed food for weight reduction diets obtained as above, and observed feed intake amounts, fat amounts and weights of the rats.

The results were that the feared appetite stimulation was not observed at all, but an appetite suppression effect was observed instead. Furthermore, the amounts of endoabdominal fats were significantly low, thus a body fat burning effect was confirmed. Moreover, although the amounts of feed intake and body fat were smaller in the rats supplied with feed having the material for processed food for weight reduction diets of the present embodiment added to it, they showed no significant difference in body weight compared to the other groups, thus suggesting that the processed food has a high use efficiency rate in the body and has the function to maintain and increase lean muscle mass such as protein and bones, and reduce only the body fat of an animal. Therefore, such a diet is not the kind of diet that also decreases lean muscle mass, leading to a degradation of one's health. Thus a possibility of dieting while maintaining and improving one's health was found.

As described above, the material for processed food for weight reduction diets according to the present invention, produced from Bonito essence is effective as a diet supplement having 100% national histidine as its main active ingredient and having the appetite suppression and fatburning effects, This suggests that it has a safe weight reduction effect without requiring rigorous exercise, providing a supplement that is promising not only for the desire to improve one's physical appearance, but also for the prevention of obesity, and, in terms of improvement, the preventive and improvement effect for life style diseases such as diabetes and high-blood pressure.

### EXAMPLE 1

An example of the present invention will be described below in accordance with accompanying drawings.

The material for processed food for weight reduction diets was obtained by the following method.

First, activated charcoal was added to Bonito Essence A-45, made by Kabushikikaisha Maruhachi Muramatsu and stirred for deodorization. After the deodorization, the activated charcoal was removed, and histidine was concentrated using reverse osmosis membrane (for concentration), followed by a desalination process using electrodialysis membrane. The resultant product was powderized to create a material for processed food for weight reduction diets,

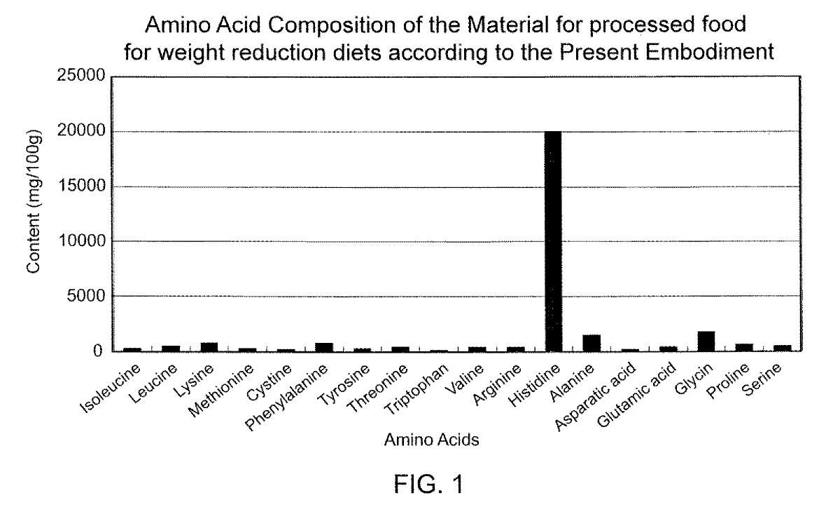

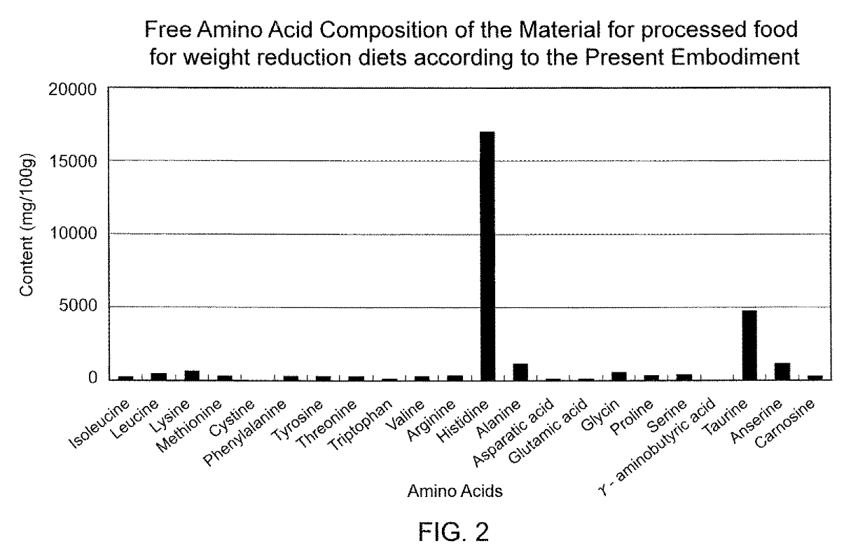

FIG. 1 is a graph showing the composition of all amino acids contained in the material. FIG. 2 is a graph showing the composition of free amino acids. In addition to histidine, Bonito essence contains taurine, BCAA (amino acid branched-chain), alanine, glycin, anserine and carnosine that are peptide compounds of alanine and histidine, and active antidiabetic ingredients such as nicotinamide that is a type of Vitamin most needed in the body and has recognized antidiabetic effects (Tryptophan-niacin metabolism in alloxan diabetic rats and partial prevention of alloxan diabetes by nicotinamide; Agric. Biol. Chem., 51(3), 811-816. 1987). Given that these components are also concentrated by the membrane compartment, Bonimax's function as dietary supplement, that is its appetite suppression and fat-burning effects are promising.

Hereafter, a case in which the material for processed food for weight reduction diets according to the present invention was applied to a feed that was subsequently tested with rats is described. In the following explanation, the material for processed food for weight reduction diets according to the present invention is referred as “BONIMAX” (product name, trademark name).

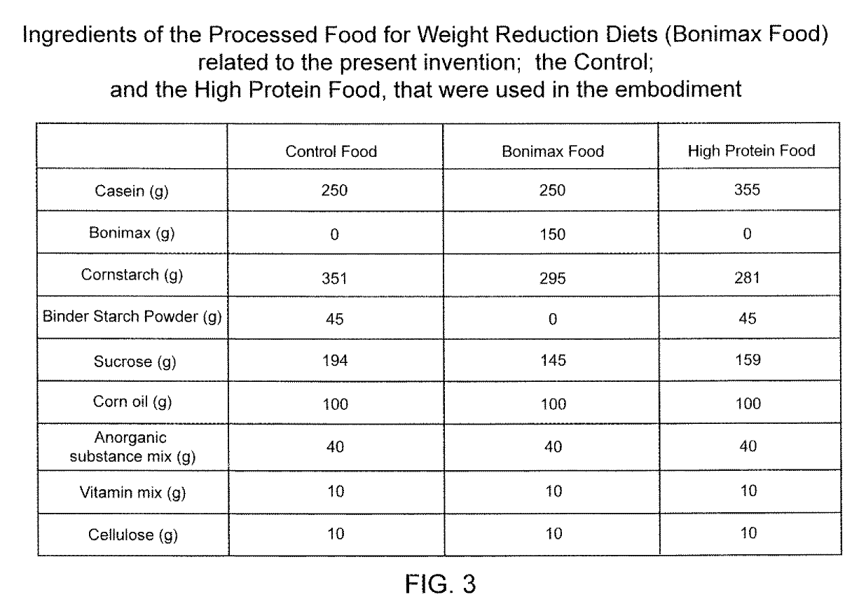

FIG. 3 shows the composition of the feed as the processed food for weight reduction diets used in this example wherein BONIMAX was added. Since the BONIMAX added feed has a higher protein content, for additional comparison reference besides the control, we added a high protein feed wherein its protein content was adjusted with casein.

Next, 15 Waitar male rats (6 week-old) were prepared for the test. The control feed, Bonimax added feed, and the high protein feed were given to respective sets of five rats, in which these feeds were freely available for the rats for three weeks, and at the end of the period, the amounts of feed intake and changes in their weight increases were examined, and subsequently through their dissection, increases in liver weight and the amounts of endoabdominal fats and the fats around the testicles were measured.

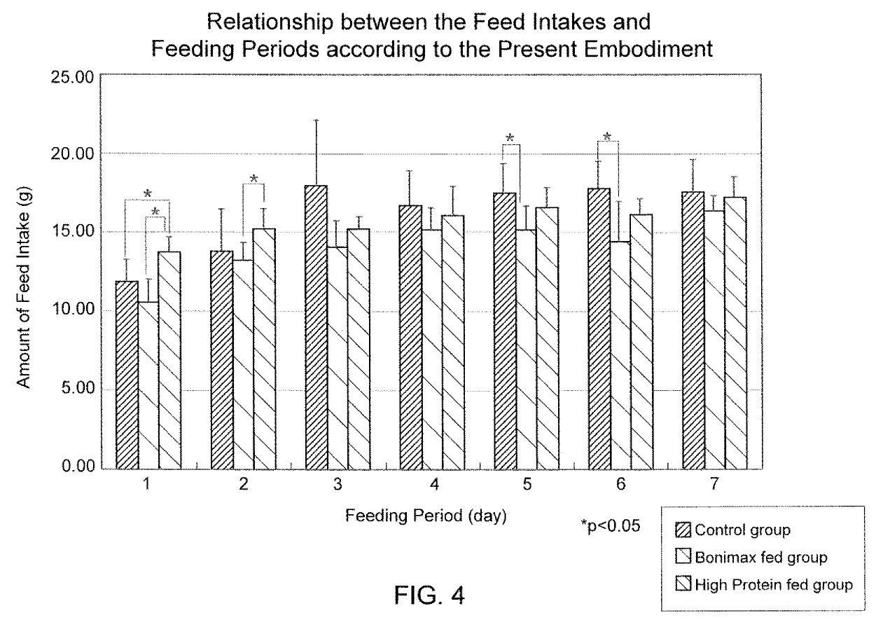

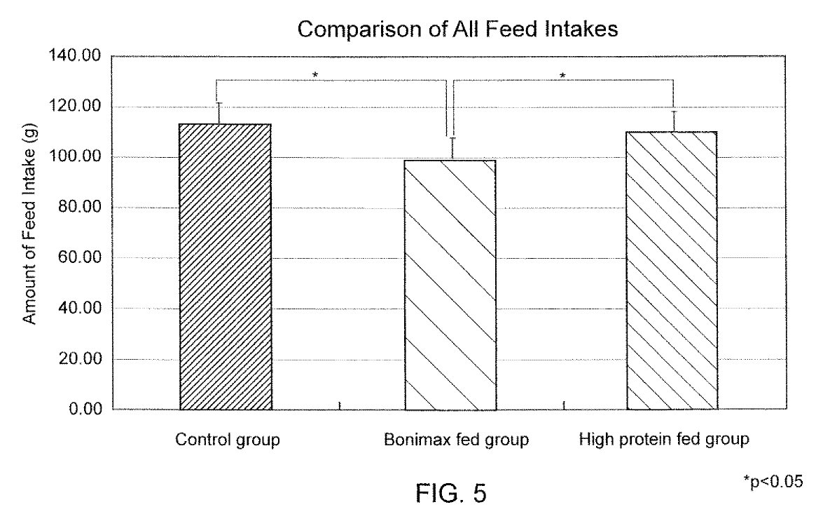

FIG. 4 is a graph showing the relationship between the feed intakes and observation periods. FIG. 5 is a graph showing all feed intake amounts. According to this graph, the feed intake amount was significantly low among the Bonimax added feed fed group, compared to the control group and the high protein feed fed group. From this data, with Bonimax where active diet components of bonito essence were condensed, the anticipated appetite stimulation was not observed at all, but an appetite suppression effect was confirmed.

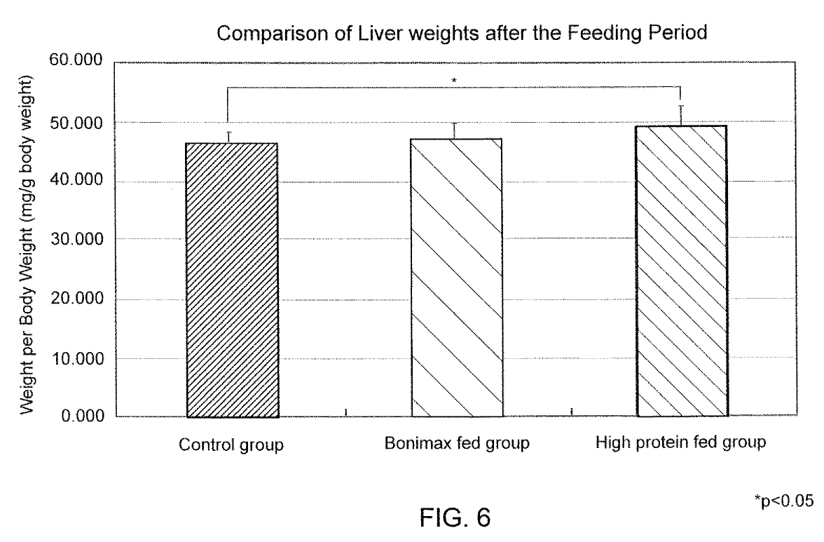

FIG. 6 is a graph showing liver weights. According to this data, the high protein group increased their liver weight significantly compared to that of the control group, showing an effect on the liver. On the other hand, the Bonimax added feed fed group did not show any significant difference to the control, thus not indicating a burden to their livers due to excessive intake of proteins.

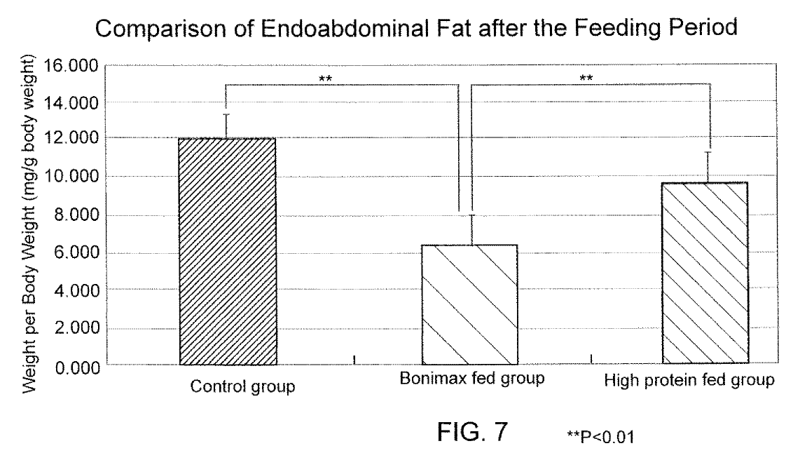

FIG. 7 is a graph showing the amount of endoabdominal fat, that is the amount of fat around abdomen. The Bonimax added feed fed group had significantly smaller endoabdominal fat, compared to the control and the high protein feed group. Since it had significantly smaller endoabdominal fat, a fat burning effect is confirmed.

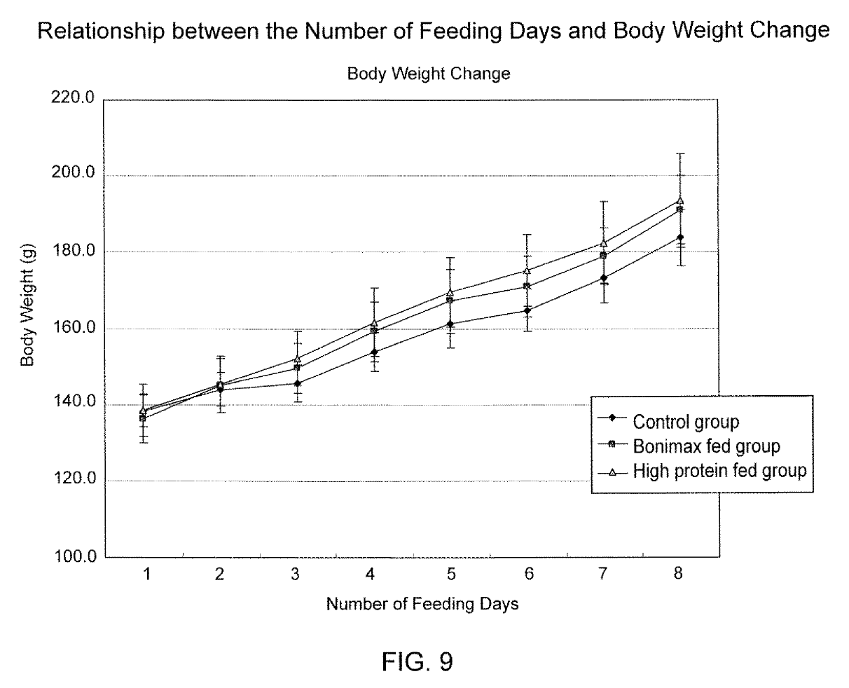

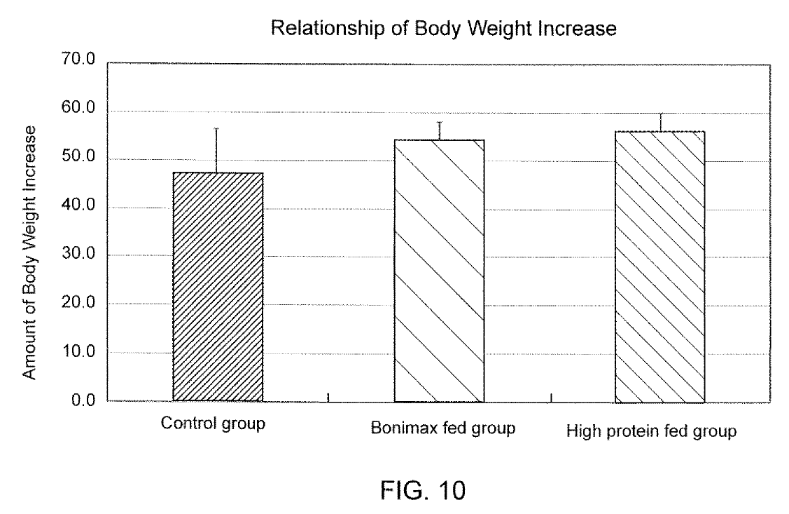

FIGS. 9 and 10 are graphs showing changes in body weight. Concerning body weight, there were no significant differences among all three groups. That is, the Bonimax added feed fed group did not have a significant difference in terms of body weight increase compared to the other two, although its feed intake amount and fat amounts were lower than the other groups, thus suggesting that Bonimax has higher bio-availability and a mechanism to increase lean muscle weight such as protein and bone, while reducing the body fat of an animal.

It should be understood that the present invention is not limited to the above described embodiment and example, and many modifications and variations of the present invention are possible without departing from the scope of the present invention. For example, in the above described example, bonito was used as the type of fish. However, it is not limited to bonito, and it can be any fish containing a good amount of histidine such as swordfish, tuna, mackerel, sardine, horse mackerel, and saury, etc.

We claim:

1. A material for processed food for weight reduction diets, comprising protein with a high histidine content, extracted from fish, to maintain body protein and reduce only the body fat of an animal (including humans).

2. The material for processed food for weight reduction diets of claim 1, wherein said active ingredients including histidine are extracted from Bonito essence by membrane concentration.

3. The material for processed food for weight reduction diets of claim 1, wherein 12,000 mg-20,000 mg weight percent of histidine is contained.

4. The material for processed food for weight reduction diets of claim. 1, being in a powder form that can be added to other foods, which is obtained from extracts from fish and processed to remove an odor and a flavor of said extracts.

5. A dietary processed food, processed using the material for processed food for weight reduction diets of claim 1.
